(19) United States
(12) Patent Application Publication
(10) Pub. No.: US 2024/0091528 A1
(43) Pub. Date: Mar. 21, 2024

(54) HYBRID SOFT-RIGID ELECTRICAL INTERCONNECTION SYSTEM

(71) Applicant: ECOLE POLYTECHNIQUE FEDERALE DE LAUSANNE (EPFL), Lausanne (CH)

(72) Inventors: Florian FALLEGGER, Lausanne (CH); Giuseppe SCHIAVONE, Morges (CH); Stephanie P. LACOUR, Etoy (CH)

(21) Appl. No.: 17/768,825

(22) PCT Filed: Sep. 16, 2020
(86) PCT No.: PCT/EP2020/075906

§ 371 Date: Apr. 13, 2022

(30) Foreign Application Priority Data
Oct. 14, 2019 (WO) PCT/EP2019/077781

(51) Int. Cl. A61N 1/05; A61N 1/36; H05K 1/03; H05K 1/09; H05K 3/10; H05K 3/38

(52) U.S. Cl. CPC A61N 1/0551; A61N 1/36125; H05K 1/0393; H05K 1/092; H05K 3/107; H05K 3/386

(57) ABSTRACT

It is disclosed an electrical interconnection system comprising: i) an interconnection board comprising an intrinsically non elastic substrate, said substrate having a first face and an opposed second face, and at least one conductive track on and/or within at least a portion of said substrate; ii) a stretchable interconnect comprising an intrinsically elastic substrate, said substrate comprising at least one well or groove comprising at least one compliant conductive element therein, said at least one well or groove being configured to accommodate said at least one conductive track of said interconnection board; and iii) at least one bolus of an electrically conductive paste located within said at least one well or groove, configured to electrically connect said at least one compliant conductive element with said at least one conductive track.

### TECHNICAL FIELD

The present invention belongs to the field of electronics and electrical devices. In particular, the present invention relates to a hybrid (soft/rigid) electrical interconnection system, methods for producing the same as well as to low-profile, hybrid (soft/rigid) multi-component electronic/electrical circuits and devices.

### BACKGROUND ART

In the field of wearables and implantable devices, soft and stretchable materials have been leveraged to produce devices that can conform to complex static and dynamic 3D shapes. Specifically, in the field of implanted neural interfaces, it has been recently shown that rigid materials conventionally used as electronic substrates, although non-toxic, give rise when implanted to inflammatory reactions due to the mechanical mismatch between the device and the soft host tissue. Using conformable, soft and stretchable materials, closer in mechanical properties to the target tissue, can mitigate this side-effects and permit safe and reliable implantation in the body.

Implantable devices consist of a substrate and encapsulation, which typically dictate the mechanical signature of the device, depending on the shape and materials used, and embedded electrical tracks and interconnections that carry the electric signal to and from the target location in a subject's body. For the substrate, several material classes can be used, such as elastomers (silicones, polyurethanes, natural rubbers, etc.), hydrogels (polymeric networks that can adsorb large quantities of water), thermosets and thermoplastics (polyimide, parylene C, etc.) and others. The embedded electrical interconnect must accompany the stretchable behaviour of the substrate and encapsulation to guarantee device functionality. This is achieved by either using intrinsically stretchable conductors (e.g. conductive polymers) or by engineering elasticity in rigid conductors (e.g. patterned metal spring structures) or embedded thin flexible interconnects in soft carriers.

Multiple independent interconnects are typically included in the device design, so that multiple channels can target different locations on the tissue. These electrical lines are usually a few tens to hundreds of microns in width and separated by gaps of the same size, with the aim of minimising the overall dimensions of the device. The typical number of parallel channels in implantable/wearable electronic devices is in the range of 8 to 128.

Due to the lack of established electronic packaging techniques that are compatible with soft materials, the ubiquitous challenge lies in the reliable connection of the numerous electric lines patterned on the soft substrates to either external hardware such as stimulators or processing units, or implanted hardware such as implanted pulse generators. The mechanics of soft substrates or carriers render them unfit for conventional connection techniques (i.e. surface mount rigid connectors, wire bonding, silicon packaging), which, if possible, would anyway stiffen the device. The most widely adopted connection solution for soft electronics relies on contacting a small wire to each individual channel on the substrate. This is both unreliable and labour intensive, and poses a significant scaling limitation to the size of the connection point (from 100 s of microns to millimetres) as the wiring process is hardly down-scalable.

International patent application WO 2017/203441 describes a system for obtaining the electrical interconnection between an intrinsically extensible conductor and a not intrinsically extensible one, or between two intrinsically extensible conductors. The system is particularly suitable for the production of devices implantable in the human or animal body, conformable and deformable, for neurostimulation and/or neurorecording. Despite the advancement in the field of hybrid soft/rigid electrical interconnects, the described interconnection system presents some drawbacks that render it not optimal for what concerns implantable devices: in particular, the alignment between the various channels, during the manufacturing steps, shall be accurately performed to assemble the various electrical channels/tracks between them, which might bring during the manufacturing process to quality issues, and eventually to electrical failures; additionally, external electrical conductors are joint to the interconnection system via known techniques such as welding, soldering, mechanical fastening or gluing with conductive glues of any kind. In a typical embodiment, the connection is made by means of through holes made on the electrical board, filled with a conductive material (e.g. tin) in which one end of conductor is embedded. This poses some issues with regards to the manufacturing burden, the re-distribution of stress forces upon elongation (strain) of the soft portion of the interconnection system, and augments the bulkiness of the entire system, which is undesirable for an implantable device.

To date, up to the inventors' knowledge, a process that is seamless in terms of foot print, reliable, scalable to a large number of channels and which prevents excessive stiffening of the device at the connection point is still lacking.

### SUMMARY OF INVENTION

In order to address and overcome at least some of the above-mentioned drawbacks of the prior art solutions, the present inventors developed a solution to seamlessly connect soft electronic interfaces with non-extensible electrical devices, such as electrical boards, having improved features and capabilities.

The purpose of the present invention was that of providing an electrical interconnection system that overcomes or at least reduces the above-summarized drawbacks affecting known solutions according to the prior art.

In particular, a first purpose of the present invention is that of providing an electrical interconnection system which is optimized in terms of size and shape to be advantageously included into thin form-factor devices, and particularly compliant biomedical devices for permanent or temporary implantation into a subject's body.

A further purpose of the present invention is that of providing an easy and reliable method for producing electrical interconnection systems having a hybrid elastic/non-elastic nature.

All those aims have been accomplished with the present invention, as described herein and in the appended claims.

In view of the above-summarized drawbacks and/or problems affecting electrical interconnection systems of the prior art, according to the present invention there is provided an electrical interconnection system according to claim 1.

Another object of the present invention relates to an article of manufacture according to claim 16.

In particular, according to the present invention an electrical interconnection system comprises:
i) an interconnection board comprising an intrinsically non elastic substrate, said substrate having a first face and an opposed second face, and at least one conductive track on and/or within at least a portion of said substrate;
ii) a stretchable interconnect comprising an intrinsically elastic substrate, said substrate comprising at least one well or groove comprising at least one conductive element therein, said at least one well or groove being configured to accommodate said at least one conductive track of said interconnection board; and
iii) at least one bolus of an electrically conductive paste located within said at least one well or groove, configured to electrically connect said at least one conductive element with said at least one conductive track.

According to an embodiment, said at least one bolus of an electrically conductive paste is substantially composed of an adhesive elastic polymer configured to mechanically connect said at least one conductive element with said at least one conductive track.

According to an embodiment, the substrate of the interconnection board is substantially composed of a flexible material.

According to an embodiment, said intrinsically non elastic substrate is planar at the interconnection site.

According to an embodiment, the at least one conductive track of the interconnection board is located on an elongated member of the intrinsically non elastic substrate.

According to an embodiment, the elongated member of the intrinsically non elastic substrate is planar.

According to an embodiment, the interconnection board comprises an array of elongated members, each comprising at least one conductive track.

According to an embodiment, said stretchable interconnect comprises an array of wells or grooves, each well or groove comprising one of said at least one conductive element therein.

According to an embodiment, said at least one well or groove is configured to entirely accommodate said at least one conductive track of said interconnection board so that said at least one conductive track is completely embedded within said at least one bolus of electrically conductive paste.

According to an embodiment, the at least one conductive element of the stretchable interconnect comprises a stretchable metallic thin film.

According to an embodiment, said at least one conductive element of the stretchable interconnect is embedded within said intrinsically elastic substrate.

According to an embodiment, said bolus of electrically conductive paste comprises a blend of a soft polymeric material and a plurality of conductive micro- or nano-particles, tubes wires and/or sheets.

According to an embodiment, an encapsulation layer of an adhesive and electrically insulating material encapsulating said opposite second face of the interconnection board and at least a portion of said stretchable interconnect including at least a portion of said at least one well or groove.

Preferably, the encapsulation layer is substantially composed of an intrinsically elastic material.

According to an embodiment, said at least one conductive track and/or said at least one conductive element comprise one end configured to be electrically connectable to an external device.

The present invention further relates to an article of manufacture comprising the electrical interconnection system as disclosed above, for instance a biomedical device configured to be temporarily or permanently implanted into a subject's body.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF DRAWINGS

FIG. 1a is a top view of an interconnection board according to one embodiment of the invention;

FIGS. 1b and 1c are cross-section views taken at different points of the interconnection board of FIG. 1a.

FIG. 2a is a top view of a stretchable interconnect according to one embodiment of the invention;

FIGS. 2b and 2c are cross-section views taken at different points of the stretchable interconnect of FIG. 2a.

FIGS. 3a to 3e and 4a to 4e schematically represents steps of a method for manufacturing the interconnection system according to the invention, and FIGS. 3f and 4f are transversal cross-sections of the systems eventually manufactured. The main difference between the embodiments represented in FIGS. 3a to 3f and 4a to 4f relate to the positioning of the conductive element, placed on (FIG. 3a) or within (FIG. 4a) the substrate;

FIG. 5a is a top view an interconnection board comprising an array of fingers, each comprising one conductive track thereon, according to an embodiment of the present invention;

FIGS. 5b and 5c are cross-section views taken at different points of the interconnection board of FIG. 5a.

FIG. 6a is a top view of a stretchable interconnect comprising an array wells or grooves, each comprising one conductive element thereon, according to an embodiment of the present invention;

FIGS. 6b and 6c are cross-section views taken at different points of the stretchable interconnect of FIG. 6a.

FIGS. 7a to 7f, 9a to 9f and 11a to 11f schematically represent different embodiments of a method of manufacturing the interconnection system according to the present invention, and FIGS. 8, 10 and 12 represents corresponding transversal cross-sections of the system eventually manufactured, wherein the system comprises an interconnection board comprising an array of fingers, each located into a corresponding well or groove of a stretchable interconnect (FIGS. 7a-7f and 8); an interconnection board comprising an array of fingers, all of them located into a single well or groove of a stretchable interconnect (FIGS. 9a-9f and 10); an interconnection board comprising an array of fingers, said fingers being located in pairs into corresponding wells or grooves of a stretchable interconnect (FIGS. 11a-11f and 12);

FIG. 13 schematically represents an electrical interconnection system according to the invention wherein at least one conductive track and/or said at least one conductive element comprise one end configured to be electrically connectable to an external device.

### DETAILED DESCRIPTION OF THE INVENTION

The subject-matter described in the following will be clarified by means of a description of those aspects which are depicted in the drawings. It is however to be understood that the scope of protection of the invention is not limited to those aspects described in the following and depicted in the drawings; to the contrary, the scope of protection of the invention is defined by the claims. Moreover, it is to be understood that the specific conditions or parameters described and/or shown in the following are not limiting of the scope of protection of the invention, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/−10% of a given value.

The following description will be better understood by means of the following definitions.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

In the frame of the present disclosure, the expression “operatively connected” and similar reflects a functional relationship between the several components of the device or a system among them, that is, the term means that the components are correlated in a way to perform a designated function. The “designated function” can change depending on the different components involved in the connection; for instance, the designated function of electrodes operatively connected with connection means is to e.g. deliver electric current to a nerve in order to electrically stimulate it. A person skilled in the art would easily understand and figure out what are the designated functions of each and every component of the device or the system of the invention, as well as their correlations, on the basis of the present disclosure.

The expression “conductive track” refers to any film, path, stripe, strand, wire or the like which is electrically conductive in nature. For the sake of clarity, the word “electrode” is herein used to mean the distal part of a conductive track which is in direct contact with a subject's tissue. However, in embodiments of the invention, the term “electrode” is used to mean both a conductive track and its distal, terminal potion configured to interface with a biological tissue. Conductive tracks according to the present disclosure are used to connect and/or close an electrical circuit, and are thus usually electrical connectors or “interconnects”. A conductive track is generally a metallic element that conducts an electric current toward or away from an electric circuit, but can be made of any suitable electrically conductive material, including but not limited to metals such as Au, Pt, Al, Cu and the like, as well as any alloy, oxides and/or combinations thereof; conductive polymeric materials; composite material such as polymeric materials embedding metal particles and/or metal strands or stripes, including insulating materials functionalized with electrically conductive flakes or fibers, for example carbon-filled polymers; liquid metals, including alloys or oxides thereof, such as gallium; electrically conductive inks; as well as any suitable combination thereof. Micro-lithography and/or micro-integrated electronics, among other techniques readily available in the art, can be adopted to fabricate the components of the electrodes.

The expressions “film” or “thin film” relate to the thin form factor of an element of the device of the invention such as a support substrate and/or a conductive track. Generally speaking, a “film” or “thin film” as used herein relates to a layer of a material having a thickness much smaller than the other dimensions, e.g. at least one fifth compared to the other dimensions. Typically, a film is a solid layer having an upper surface and a bottom surface, with any suitable shape, and a thickness generally in the order of nanometers, micrometers or even millimetres, depending on the needs and circumstances, e.g. the manufacturing steps used to produce it. In some embodiments, films according to the invention have a thickness comprised between 1 nm and 10 mm, such as between 1 nm and 10 nm, 20 nm and 100 nm, 5 μm and 5 mm, between 5 μm and 1 mm, between 10 μm and 1 mm, between 5 μm and 500 μm, between 50 μm and 500 μm between, between 50 μm and 150 μm, 100 μm and 500 μm or between 200 μm and 500 μm. When referring to thin electrode films, those can have a thickness comprised between 1 nm and 500 μm, such as between 20 nm and 200 nm or between 50 nm and 100 nm. These dimensions are considered to be optimal in the frame of the present invention for what concerns stretchability and mechanical compliance of the device meant to be interfaced with body tissues

The term “compliant”, when referred to a conductive element such as an electrode, track and/or interconnect, refers to the behaviour of said conductive element to adapt to change its shape according to the shape change of the support it adheres to, without substantially compromising mechanical and/or electrical performances. The term “compliant” is intended to include any conformable structure which is compressible, reversibly compressible, elastic, flexible, bendable, stretchable or any combination thereof. Examples of compliant electrodes known in the art include metal thin-films (including patterned electrodes, out-of-plane buckled electrodes, and corrugated membranes), metal-polymer micro/nano-composites, carbon powder, carbon grease, conductive rubbers or conductive paints, a review of which is provided in Rosset and Shea (Applied Physics A, February 2013, Volume 110, Issue 2, 281-307), incorporated herein in its entirety by reference. As it will be apparent to those skilled in the art, built-in multilayers or stacks of several layers of any of the above polymeric, composite, metallic and/or oxide materials, as well as combinations thereof, are encompassed in the definition of compliant interconnect. Preferably, but not limited to, the electrodes, tracks and/or interconnects according to the invention are compliant in nature. Preferably, but not limited to, the electrodes, tracks and/or interconnects according to the invention are stretchable in nature. In some embodiments, stretchable electrodes as the ones described in International Patent Applications WO 2004/095536, WO 2016/110564 and/or WO 2018/100005A1, incorporated herein in their entirety by reference, can be used.

As used herein, the term “stretchable” refers to the elastic behaviour of an item. In particular, a stretchable item can withstand an elongation or multidirectional strain, upon a single or multiple cycles, comprised between 1 and 500%, preferably at least 5%, such as about 50%, about 100% or about 200%, of its size at rest without cracking or loss of its physical and/or mechanical properties, which represents an advantage in those contexts and/or body structures in which several cycles of mechanical stresses over time can be foreseen.

In the frame of the present invention, “physical and/or mechanical properties” means, by way of examples, stress-strain behaviour, elastic modulus, fracture strain, conformability to curvilinear surfaces, compliance to soft surfaces, thickness, area and shape which, in a set of embodiments according to the invention, have to be as similar as possible to those to be found in tissues of a subject's body.

Within the frame of the present invention, the expression “intrinsically not elastic material” has to be understood as meaning a material which, once subjected to strain (pressure, stress, stretching, distorsion or the like) either breaks or is deformed permanently, i.e. without gaining again in a spontaneous and/or natural way, its original shape and dimensions. To the contrary, an “intrinsically elastic material” is a material which, once subjected to strain gains again in a spontaneous and/or natural way its original shape and dimensions.

The term “subject” as used herein refers to animals, including birds and mammals. For example, mammals contemplated by the present invention include human, primates, domesticated animals such as cattle, sheep, pigs, horses, laboratory rodents and the like.

As used herein, “treatment” and “treating” and the like generally mean obtaining a desired physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it for example based on familial history, overweight status or age; (b) inhibiting the disease, i.e., arresting its development; or relieving the disease, i.e., causing regression of the disease and/or its symptoms or conditions such as improvement or remediation of damage. The term “diagnosis”, “diagnostic” and the like refers to identifying the presence or nature of a pathological condition in a subject.

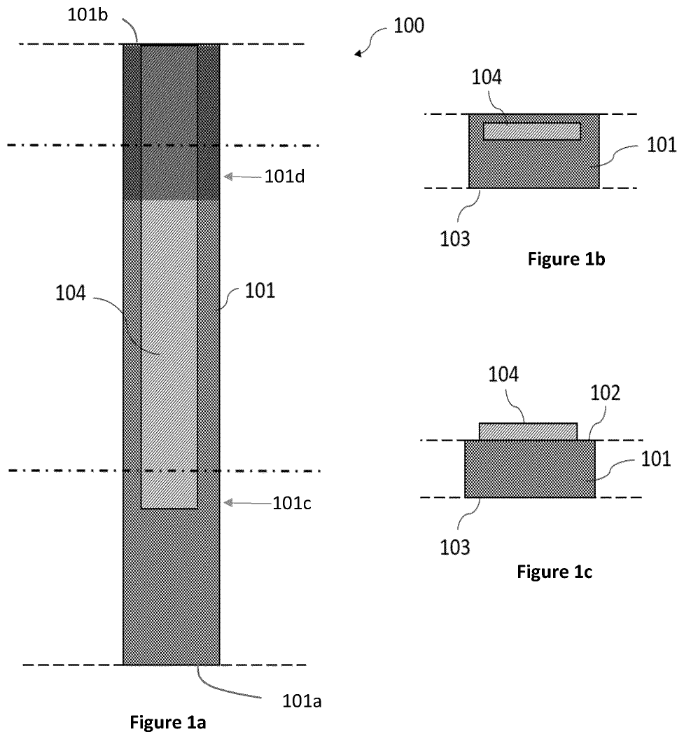

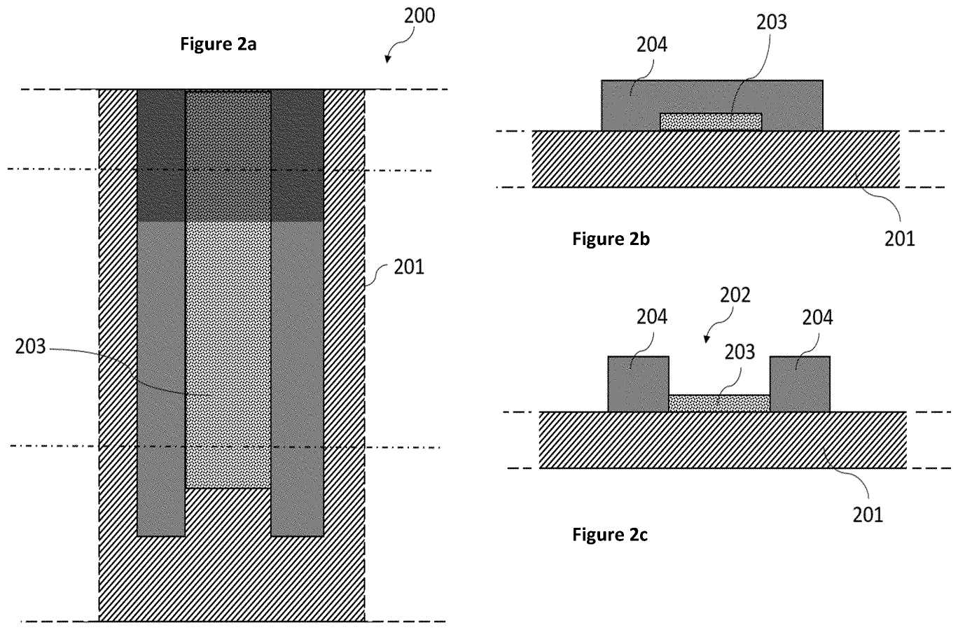

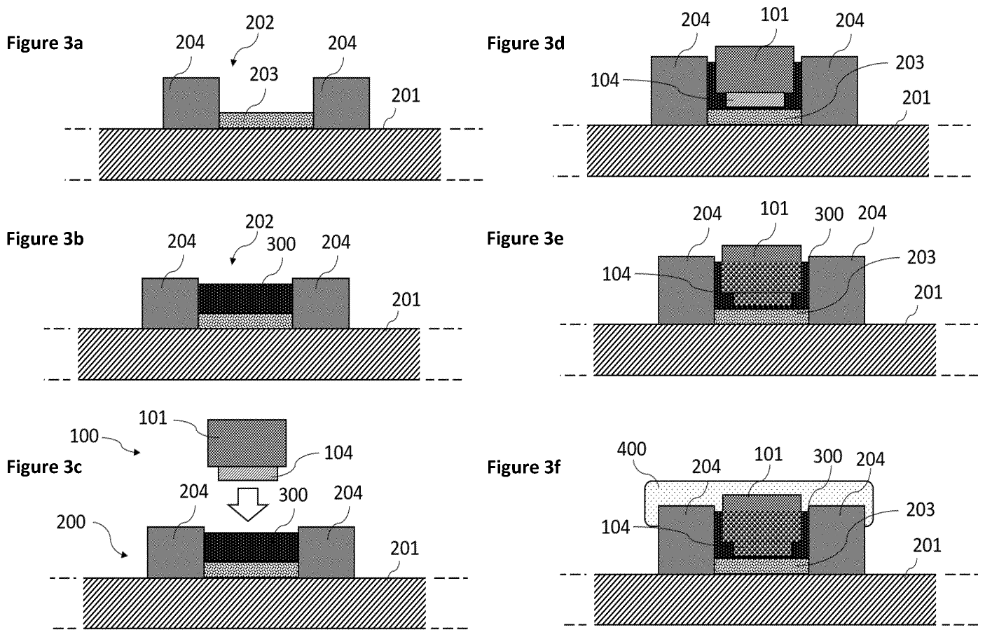

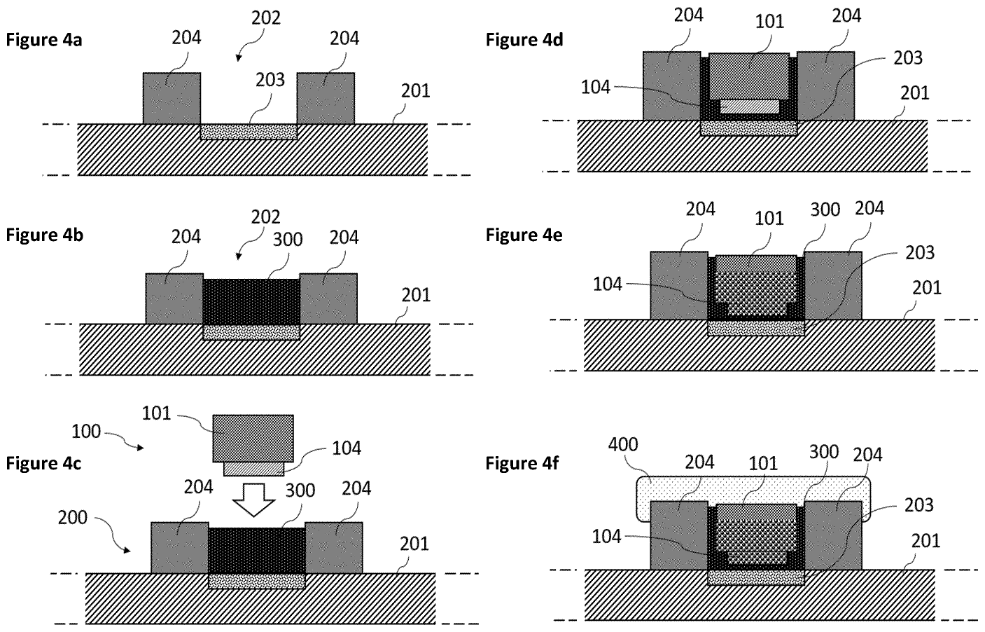

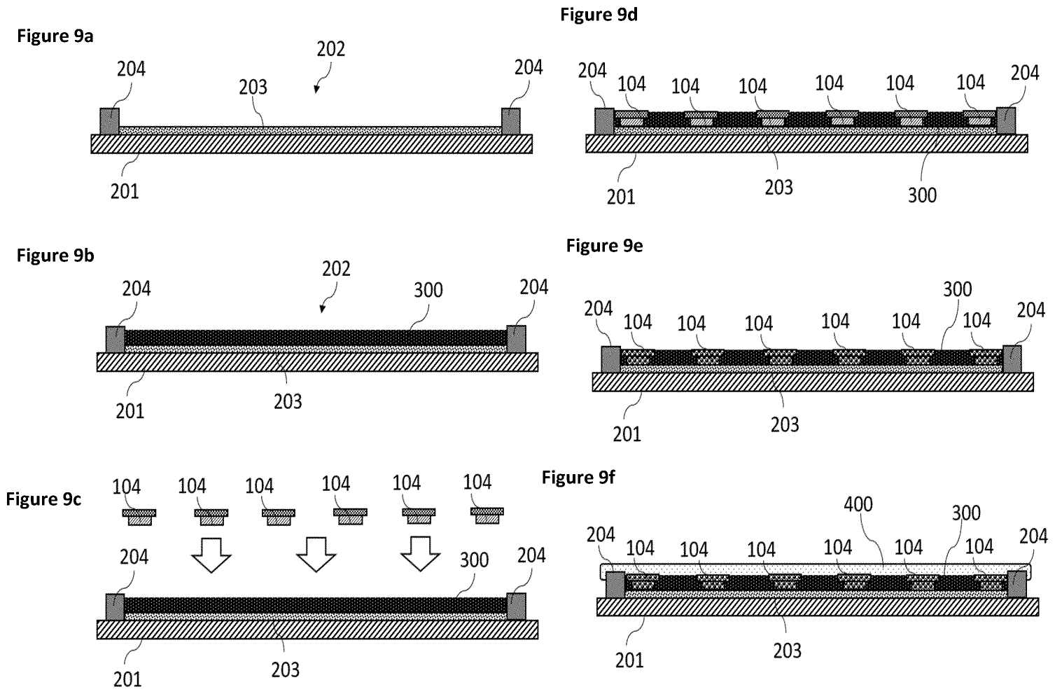

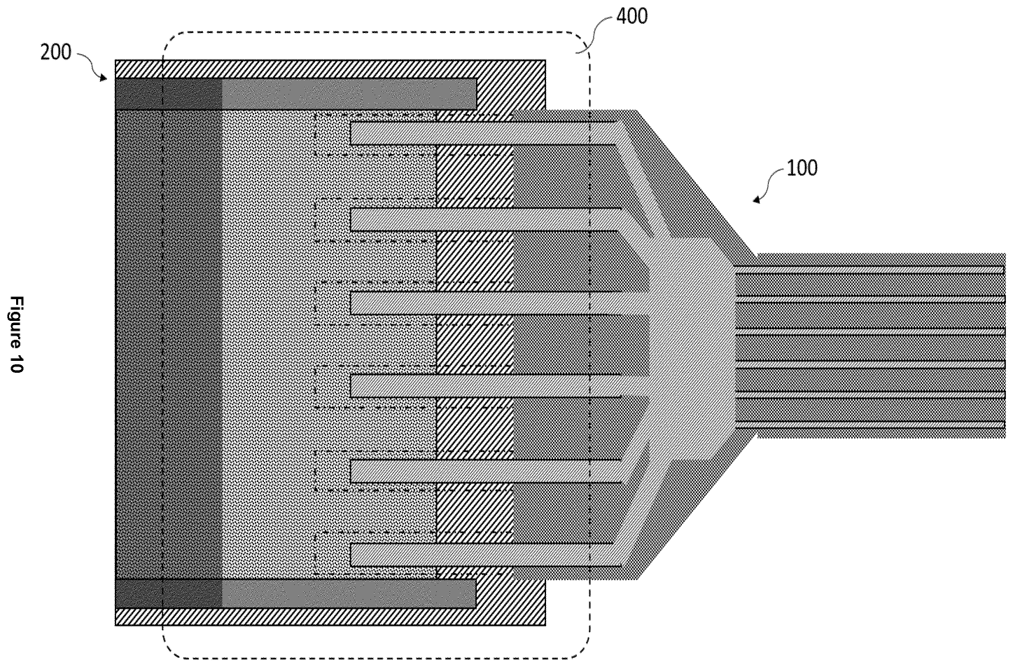

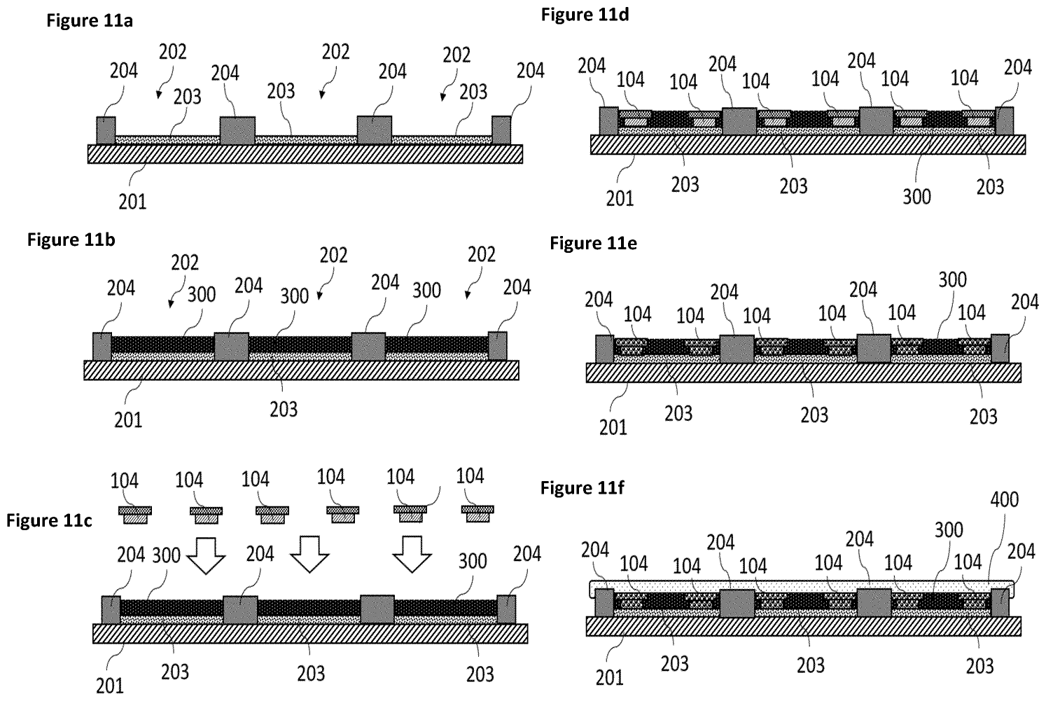

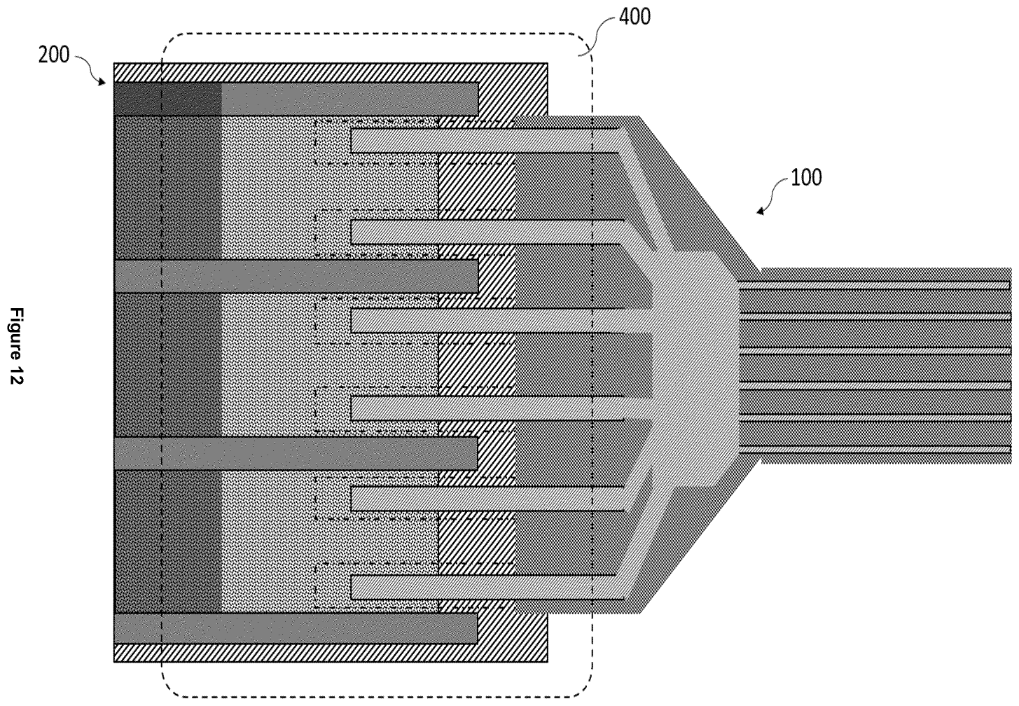

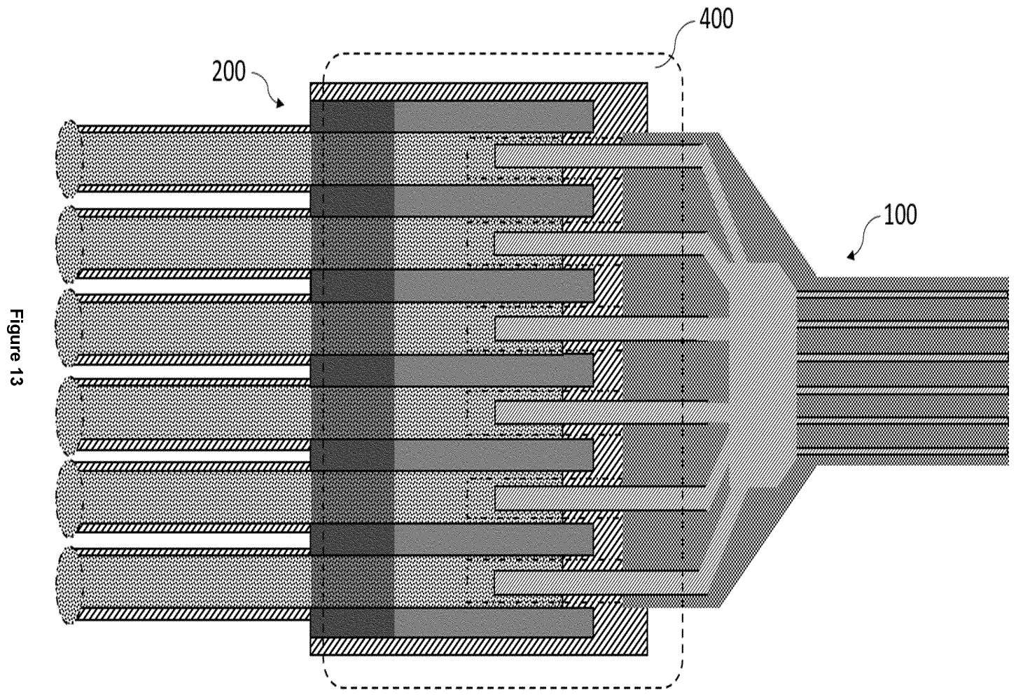

With reference to FIGS. 1 to 12, the invention features an electrical interconnection system comprising:
i) an interconnection board 100 comprising an intrinsically non elastic substrate 101, said substrate 101 having a first face 102 and an opposed second face 103, and at least one conductive track 104 located on or within at least a portion of said substrate 101 (FIGS. 1a to 1c);
ii) a stretchable interconnect 200 comprising an intrinsically elastic substrate 201, said substrate 201 comprising at least one well or groove 202 comprising at least one conductive element 203 therein, said well or groove 202 being configured to accommodate said at least one conductive track 104 of said interconnection board 100 (see for example FIGS. 2 to 4); and
iii) at least one bolus 300 of an electrically conductive paste located within said well or groove 202 and configured to electrically connect said conductive element 203 with said interconnection board conductive track 104 (see for example FIGS. 3a to 3f or 4a to 4f). In embodiments of the invention, the system further comprises an encapsulation layer 400 of an adhesive and electrically insulating material located on both the second face 103 of the interconnection board 100 and the stretchable interconnect 200 (see for example FIGS. 3a to 3f or 4a to 4f).

For instance, with reference to FIG. 3f, the interconnection board 100 is accommodated in the well or groove 202 with the first face 102 towards the substrate 201, i.e. towards the bottom of the well or groove 202, with the conductive track 104 faced to the conductive element 203. The opposite second face 103 of the interconnection board 100 is arranged at an aperture of the well or groove 202, for instance protruding from the aperture. The encapsulation layer 400 encapsulates the opposite second face 103 of the interconnection board 100 with at least a portion of the well of groove 202 at the aperture thereof, so as to close the interconnection board 100 in the well or groove 202. A remaining portion of the well or groove 202 may be free from the encapsulation layer 400.

The substrate 101 of the interconnection board 100 may have a predetermined length extending from one side 101a to an opposite side 101b along which the first surface 102 and the second surface 103 are arranged. The substrate 101 may have different thickness at portions 101c, 101d thereof arranged at different distance from the one lateral side 101a and the conductive track 104 may be exposed out from the first surface 102 at one of said portion 101c less thick than another portion 101d where the conductive track 104 is not exposed.

In order to address the drawbacks affecting the prior art, one of the key inventive concepts characterizing the system of the invention relies in the presence, on the intrinsically elastic substrate 201 of the hybrid elastic/non-elastic electrical interconnection system, of individual electronic contact implemented as wells or grooves 202 comprising at least one conductive element 203 therein, said wells or grooves 202 being patterned in the elastic substrate 201. By adjusting, in a preliminary planning phase, the design of the conductive track 104 located on the intrinsically non elastic substrate 101 of a mating interconnection board 100 to match the size (width and gap) of the wells or grooves 202, the conductive track 104 can be easily placed on the elastic device substrate, as it self-aligns to the walls 204 of the well 202 structures. Advantageously, where more than one conductive track 104 is present, in one embodiment each conductive track 104 rests inside the wells 202 and are isolated from one another by the well walls 204.

A further advantage of the design proposed by the present invention is the remarkable reduction in the bulkiness of the interconnection system, particularly in terms of thickness: as the conductive tracks 104 of the interconnection board 100 are eventually located within the matching wells or grooves 202 of the stretchable interconnect 200, the interconnection board 100 and the interconnect 200 result coplanar and not coupled in a stacked configuration. The so-obtained arrangement allows to significantly reduce the size of the entire system compared to solutions known in the art, creating a seamless hybrid soft/rigid interconnection. Without being bond to any theory, it is deemed that the proposed configuration further allows a more homogeneously distribution of the strain upon an elongation stress along the stretchable portion of the electrical interconnection system, thus reducing the risks of failure due to breakage.

The stretchable interconnect 200 may be manufactured with methods known in the art such as microfabrication and photolithography, as will become apparent in the following description. An intrinsically elastic substrate 201 is first provided on a temporary substrate such as a rigid silicon wafer. Substrate 201 is substantially composed of a soft polymeric matrix made of a soft polymeric material, or combinations of many soft polymeric materials, possibly biocompatible ones, whenever needed, to fit with biomedical applications. The term “soft” is herein meant to include any material that is compressible, reversibly compressible, elastic, flexible, stretchable or any combination thereof. Particularly, a soft material includes materials having a small Young's module (typically of <100 MPa, such as between 0.01 and 100 MPa), providing a large elongation upon a strain stress, typically of >5% of the elongation of a soft structure at rest. In such a way, the obtained device is highly compliant even for thickness of several millimeters to centimeters upon experiencing a deformation.

In preferred embodiments of the present invention, soft materials are stretchable, i.e. elastically deformable upon elongation, preferably in more directions. The stretchability of the support 201 is provided by the materials this is substantially composed of; in this context, in preferred embodiments the support substrate 201 is substantially made of a soft polymeric material, or combinations of many soft polymeric materials, possibly biocompatible ones, or polymeric materials coated with soft polymeric material or hydrogels, or made of composite materials. Examples of suitable materials for the soft polymeric matrix composing the substrate 201 are for instance thermosets or thermoplastics such as styrene butadiene styrene (SBS) or styrene ethylene butylene styrene (SEBS), soft foams such as polyurethanes including reticulated polyurethanes, polyvinyl chloride (PVC), neoprene, uncrosslinked neoprene, cross-linked polyethylene, polyether, ethylene-vinyl acetate (EVA), polyethylene-vinyl acetate (PEVA), polypropylene glycol (PPG), latex, elastomeric materials such as silicone rubber (e.g. polydimethylsiloxane PDMS) or fluorosilicone rubber, thermoplastic elastomers such as styrenic block copolymer (SBC), ethylene propylene diene monomer (EDPM) rubber, butyl rubber, nitrile rubber, or combinations of any of the foregoing.

Accordingly, the support 201 has, in preferred embodiments of the invention, a Young's modulus comprised between about 1 kPa and 1 GPa, such as for instance between about 100 kPa to about 1 GPa, between about 100 kPa to about 1 GPa, between about 5 MPa to about 1 GPa, between about 100 kPa to about 100 MPa, between about 100 kPa to about 5 MPa, between about 10 kPa to about 300 kPa or between about 10 kPa to about 10 MPa, preferably between about 1 MPa to about MPa, which are suitable ranges of values matching the Young's modulus of many biological tissues and surfaces to avoid mechanical mismatches between said tissues and a biomedical device, and/or for mimicking physical and/or mechanical properties of bodily tissues.

In a second step, at least one conductive element 203 is provided on the substrate 201. By way of example, conductive element 203 may be provided on at least one surface of a cured soft and stretchable elastomeric material such as PDMS by deposing a metal such as Au, Pd, Pt, Ir or alloys thereof via e.g. physical vapour deposition such as thermal evaporation or sputtering, chemical vapour deposition, spray coating, lamination, Cluster ion implantation or Supersonic Cluster Beam Implantation. The term “curing” is herein used to refer to the toughening or hardening of a polymer material by cross-linking of polymer chains, brought about by electron beams, heat, and/or chemical additives such as crosslinkers, as well known to a person skilled in the art. When the additives are activated by ultraviolet radiation, the process is also called UV cure. In this non-limiting and combinable embodiment, the at least one conductive element 203 comprises or consists of a stretchable metallic thin film having a thickness comprised between 10 and 80 nm, with track width comprised between 50 and 300 μm.

Additionally or alternatively, said at least one conductive element 203 may be substantially composed of composite materials such a metallic and/or carbon-based inks and pastes deposited on the cured soft material surface by e.g. spray coating, sputtering, screen printing or inkjet printing. The composite material(s) may alternatively be composed of a soft polymeric matrix “doped” or embedding micro or nanoparticles such as carbon nanotubes or micro/nanoparticles, gold micro/nanoparticles, platinum micro/nanoparticles and the like.

Additionally or alternatively, said at least one conductive element 203 may be substantially composed of liquid metals or alloys thereof, preferably one of gallium and a gallium-based alloy, deposited on the cured soft material surface by e.g. physical vapour deposition, chemical vapour deposition, spray coating, thermal evaporation/condensation, direct writing screen printing, doctor blading or inkjet printing. Combinations of any of the above solutions are also envisageable.

In a third step, at least part of the conductive element 203 is encapsulated into the same or a different soft matrix substantially composed of a soft polymeric material. To this aim, a soft curable material is provided in such a way to embed the conductive element 203 according to a method known in the art such as overmolding, spray coating, dispensing (pouring), forming, compression molding, dip coating and the like. Preferably, the conductive element 203 is encapsulated into the same or a different soft matrix, and this latter is subsequently patterned by e.g. photolithography in order to 1) expose through vias the conductive element 203 and 2) create wells or grooves 202 delimited by walls 204 resulting from the patterning process. The result at the end of those steps is a substrate 201 comprising at least one well or groove 202 comprising at least one conductive element 203 therein: the encapsulation thus covers the substrate everywhere except on one end of the stretchable interconnect 202, where a “pad” from the conductive element 203 is used for electrical contact. The dimensions of the well or groove 202 is chosen in order to accommodate at least one conductive track 104 of an interconnection board 100, as will be detailed later in the description.

According to one embodiment, exemplarily depicted in FIG. 4a to 4f, the at least one conductive element 203 of the stretchable interconnect 200 is embedded within the intrinsically elastic substrate 201. Preferably, conductive elements 203 embedded within the intrinsically elastic substrate 201 are composed of a soft polymeric matrix doped with or embedding micro or nanoparticles such as carbon nanotubes or micro/nanoparticles, gold micro/nanoparticles, platinum micro/nanoparticles and the like.

As for the stretchable interconnect of the invention, an interconnection board 100 according to the present disclosure can be manufactured with methods known in the art. Interconnection board 100 according to the invention comprises an intrinsically non elastic substrate 101 and at least one conductive track 104 located thereon, for instance on at least a first face 102 and/or on an opposed second face 103. Additionally or alternatively, said conductive track 104 can be embedded within said substrate 101 and exposed or partially exposed through vias, possibly metallized vias. The conductive track(s) 104 may be passivated on at least a portion thereof with the same material composing the substrate 101 or a different one, and vias can be opened to access the conductive portion of the track(s) 104. In some preferred embodiments, the substrate 101 of the interconnection board 100 is substantially composed of a flexible material. In this context, the term “flexible” refers to a bendable, intrinsically non-elastic material such as for instance plastics, thermoplastics e.g. polyimide or parylene, liquid crystal polymer (LCP), thin fiber glass composites in epoxy (e.g. FR4) and similar. A flexible substrate 101 is of particular interest for interconnection systems for implementation into biomedical implant/devices such as neural interfaces, as it may reduce the mechanical mismatch between the device parts and the bodily tissues such as e.g. the cortex, while at the same time being compliant and sufficiently resistant to avoid risks of mechanical failure.

Preferably, and still within the scope of the present invention, said interconnection board intrinsically non elastic substrate 101 is planar, at least at the interconnection site, that is, at least the portion of the substrate the conductive track 104 lies on, and which is functional to establish the electrical connection with the partner portion of the stretchable interconnect 200. This configuration allows the possibility to reduce the form factor of the final assembly in terms of thickness, i.e. once the at least one conductive track 104 is coupled with the stretchable interconnect 200 such that the track 104 lies within the assigned well or groove 202. According to some embodiments, moreover, the stretchable interconnect 200 is planar.

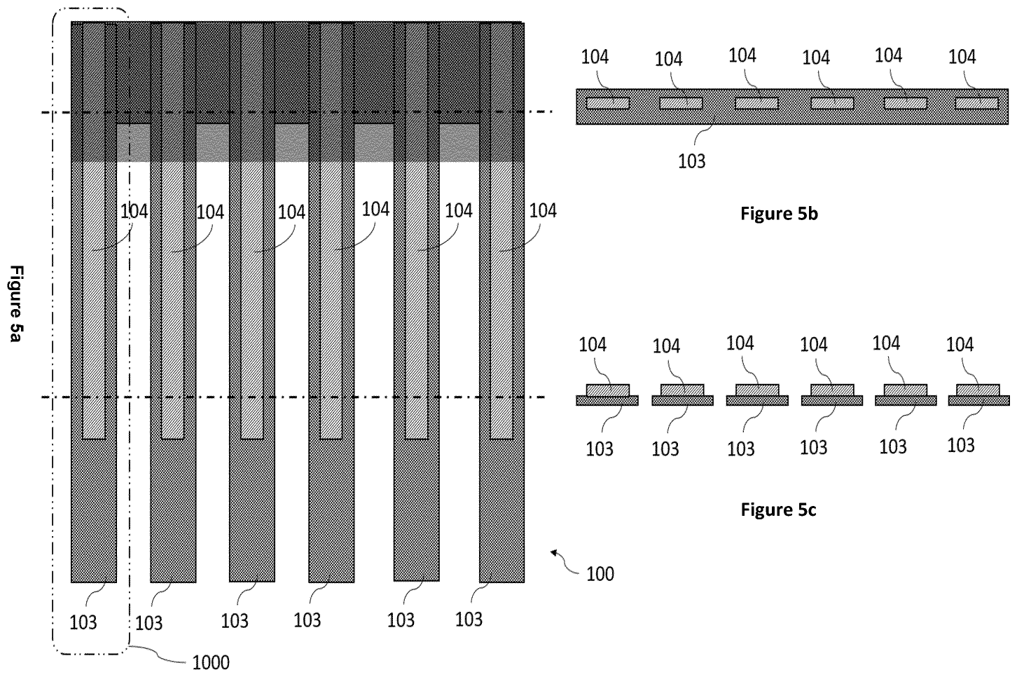

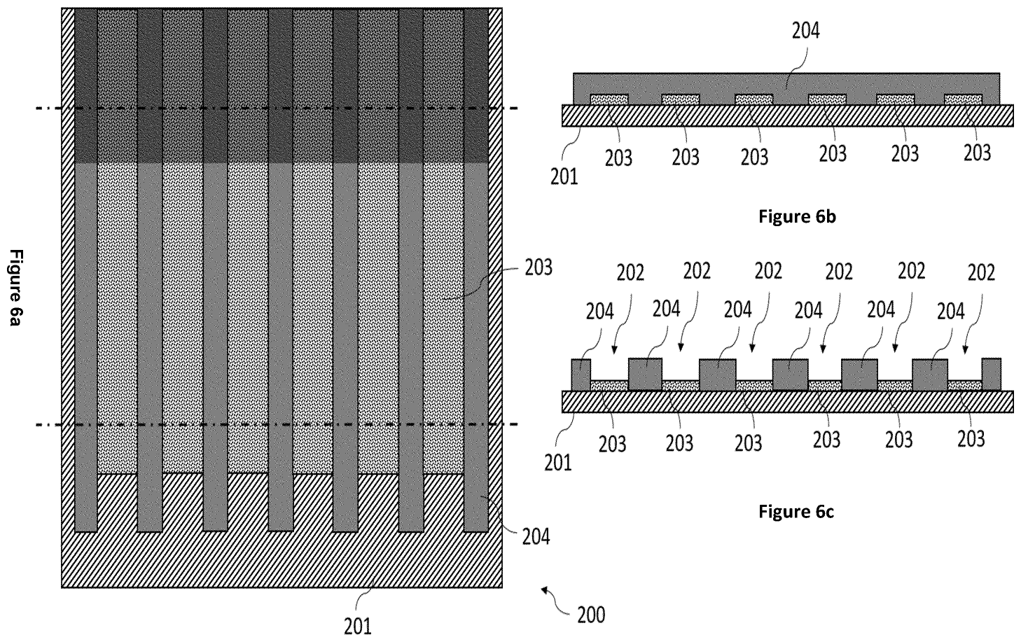

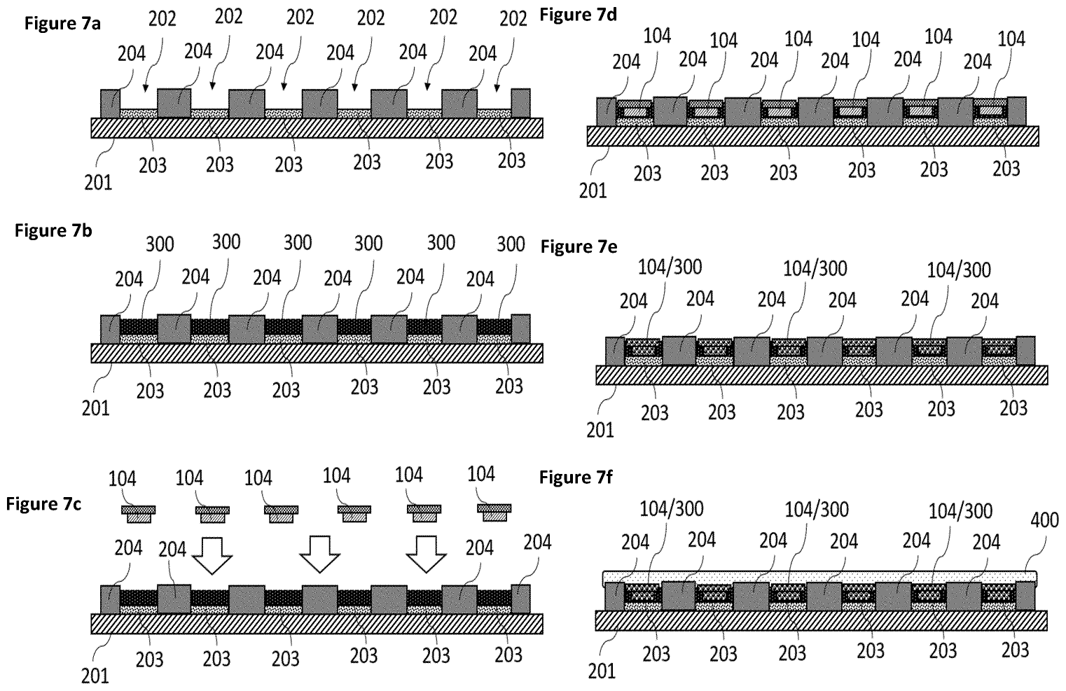

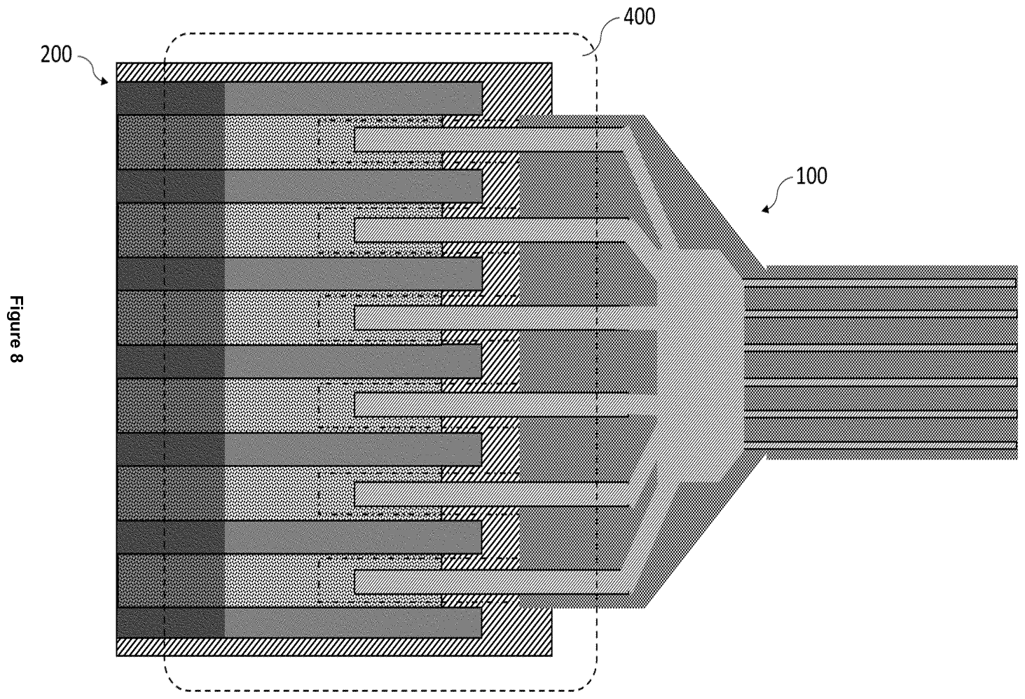

In embodiments, the at least one conductive track 104 of the interconnection board 100 is located on an elongated member 1000, which is possibly a planar elongated member, of the intrinsically non elastic substrate 101. A plurality or array of elongated members 1000, each comprising at least one conductive track 104 thereon/therein, is contemplated in embodiments of the invention (FIGS. 5a to 5c), said array possibly allowing to multiplex the functionalities of the final interconnection system, providing a plurality of separated channels. An array of elongated members 1000 might be referred to hereinafter as “fingers”. According to the above embodiment, the stretchable interconnect 200 comprises a substrate 201 having a plurality or array of wells or grooves 202 comprising at least one conductive element 203 therein, the wells 202 being configured to accommodate the matching conductive tracks 104 (FIGS. 6a to 6c), depending on the needs and circumstances. Some exemplary embodiments of array configurations, as well as of interconnection systems according to the invention obtainable therefrom, are depicted in FIGS. 7a, 7b, 7c, 8, 9a, 9b, 9c, 10, 11a, 11b, 11c and 12.

With reference to FIGS. 3a to 3c and 4a to 4c, two non limiting embodiments of one method of manufacturing of the interconnection system according to the invention is shown, as well as one embodiment of said system depicted in a transversal cross-section. The main difference between the embodiments shown in FIGS. 3a-3c and 4a-4c relates in the positioning of the conductive element 203, placed on (FIG. 3a) or within (FIG. 4a) the substrate 201. In a first step (FIG. 3a or 4a), a stretchable interconnect 200 is provided, better shown as a top view in FIG. 2a.

In a second step (FIG. 3b or 4b), at least one bolus 300 of an electrically conductive paste is located within the well or groove 202. The bolus 300 of an electrically conductive paste is configured to electrically connect the conductive element 203 with an interconnection board conductive track 104. In some embodiments, said at least one bolus 300 of an electrically conductive paste is substantially composed of an adhesive elastic polymer configured to mechanically connect said conductive element 203 with said interconnection board conductive track 104. This configuration facilitates the mechanical link of the different elements composing the system, thus reducing mechanical mismatches between the “soft” and the “rigid” components of the final assembly.

In one embodiment, said bolus 300 of electrically conductive paste comprises a blend of a soft polymeric material and a plurality of conductive micro- or nano-particles, tubes wires and/or sheets. Typically, said elements are composed of a metallic material selected from silver (e.g. silver powder), gold, platinum and the like micro- or nano-particles, tubes wires and/or sheets, as well as oxides and/or combinations thereof, carbon powder, carbon nanotubes, graphene nanosheets etc.

In a third step (FIGS. 3c to e or FIGS. 4c to e), the intrinsically non elastic substrate 101 of the interconnection board 100 comprising conductive track 104 is placed within a receiving well 202 in a way as to establish a solid physical and electrical connection with the stretchable interconnect 200. To this aim, the conductive track 104 is embedded into the bolus 300 of electrically conductive paste that, depending on the needs and circumstances, can be substantially composed of an adhesive elastic polymer configured to mechanically connect the conductive element 203 of the stretchable interconnect 200 with said interconnection board conductive track 104. The adhesive elastic polymer of the bolus 300 can be in a liquid or semi-solid form in a first time, and subsequently cured by means known in the art such as photopolymerization, chemical polymerization, heat (e.g. cured in an oven at 80° C. for one hour) and the like, once the conductive track 104 is in plane within the well 202. The bolus 300 might include reactive chemical species acting as cross-linking agents (e.g. photoinitiators) to help, speed up and/or enhance the curing process. Accordingly, in the frame of the present disclosure, a “paste” includes also soft solid materials which result from a curing process of (an) initially non-soft solid precursor(s). In one embodiment, said at least one well or groove 202 is configured to entirely accommodate said at least one conductive track 104 of said interconnection board 100 so that said at least one conductive track 104 is completely embedded within said at least one bolus 300 of electrically conductive paste. In other embodiments, the at least one well or groove 202 is configured to entirely accommodate said at least one conductive track 104 as well as the intrinsically non elastic substrate 101 comprising the same.

In a last, optional step (FIG. 3f or 4f), the electrical interconnection system is encapsulated with an encapsulation layer 400 of an adhesive and electrically insulating material located on both the second face 103 of the interconnection board 100 and the stretchable interconnect 200. In order to maintain as much as possible flexibility and stretchability of the final assembly, in compliance with the general spirit of the invention, said encapsulation layer 400 is preferably substantially composed of an intrinsically elastic material, for instance an elastomeric material such as silicone rubber, polybutyl rubber, polyurethane, thermoplastic vulcanizates, etc. The encapsulation layer 400 guarantees not only mechanical robustness to the entire system, but also avoids risks of shortcuts with the surrounding environment and/or between the several components of the system.

In preferred embodiments, in the electrical interconnection system according to the invention said at least one conductive track 104 and/or said at least one conductive element 203 comprise one end is configured to be electrically connectable to an external device (FIG. 13). As it will be apparent, being the system of the invention an “electrical interconnection” system, the elements composing thereof are supposed to create a suitable electrical connection between at least two elements. For reasons that will become apparent in the following description, both elements might be electrical, electronic or electro-mechanical external devices, or one of those elements might be a tissue, organ or otherwise a part of a subject's body, as in the case of biomedical devices for bodily interface.

As will be apparent to a person skilled in the art, the interconnection system of the invention can be used and implemented to connect “soft” and “rigid” components of systems, devices and the like. Therefore, one aspect of the invention relates to an article of manufacturing comprising the electrical interconnection system as described herein. The articles of manufacturing that may enjoy of the invention herein described includes medical and biomedical devices including implantable devices, wearable devices such as “smart” garments (t-shirts, caps, shoes and the like embedding electronic components), wristbands, thin form factor items such as (flexible) displays, furniture embedding electronic components such as chairs, and so forth.

Particularly, as anticipated, in the frame of the present invention the electrical interconnection system can be advantageously used and incorporated into biomedical devices, particularly devices configured to be temporarily or permanently implanted into a subject's body. The term “subject” as used herein refers to mammals or even birds. For example, mammals contemplated by the present invention include human, primates, domesticated animals such as cattle, sheep, pigs, horses, laboratory rodents and the like. Within the meaning of the present invention, a “fixed implant” defines a biomedical device having the ability to conform to established and/or customised surgical procedures and to reside in vivo without producing adverse biological reactions over extended periods of time, such as for instance over 7 days. Still within the meaning of the present invention, a “removable implant” defines a biomedical device having the ability to conform to established and/or customised surgical procedures and to reside in vivo for a limited amount of time, such as for instance the time of a surgical operation.

Advantageously, in the frame of a biomedical device, the electrical interconnection system of the invention may allow mainly to 1) reduce the form factor of a device including the same, 2) optimize the distribution of the mechanical stress along the device upon a deflection stimulus (e.g. expansion, contraction, bending, torsion, twist, linear or area strain) and 3) better comply with the mechanical properties of the surrounding biological tissue, reducing mechanical mismatches and therefore adverse reactions (e.g. inflammation or fibrotic responses) in a subject.

Biomedical devices according to the invention can be used for sensing, measuring and/or monitoring physiological and/or physiopathological parameters in a subject in needs thereof, with the aim of treating the same. Advantageously, the interconnection system of the invention can be implemented into devices which are susceptible to undergo physical and/or mechanical stresses such as deflections upon implant. For examples, neural interfaces for the treatment of disorders of the Central Nervous System and/or the Peripheral Nervous System, including are typically included into a group of devices according to this aspect of the invention. Alternatively, electrode array implants that aim at interfacing with the surface of soft tissues such as the heart, liver, gut, bladder, retina and the like are also included into to this aspect of the invention. As a way of example, microelectrode arrays are particularly suitable to be used as a neural interface with the spinal cord, brain or peripheral nerves or soft biological tissue, for instance for the purpose of stimulating and/or recording neurological or cardiac activity, as well as for monitoring hippocampal electrical activity after traumatic brain injury or bladder afferent activity, or even for stimulating electrical potential of excitable cells or the like, and may advantageously enjoy of the interconnection electrical system of the invention.

### Example

In an implemented, non-limiting example according to the invention, an interconnection electrical system has been manufacture and included into a biomedical device.

A comb structure 100 implemented as a flexible PCB (FPCB), comprising a plurality of fingers 1000 having each a conductive track 104 made of a thin film of a metallic material, has been placed on a soft interconnect 200 so that each finger 1000 lies in its assigned channel 202: the fingers of the comb automatically self-align to the walls of the well structures on the device and are isolated from one another by the well walls.

The channel 202 is filled with a conductive paste 300 before or after the comb is placed, for instance by stencil printing, to provide an intrinsically elastic electrical contact between the flexible structure 100 and the stretchable interconnect 200 on the device. Once placed, the FPCB is secured in place and isolated electrically by a silicone sealant 400 to lock the components in place and provide mechanical stability with respect to the substrate. As the fingers 1000 are not rigidly connected to the soft substrate 200, the assembly can maintain elasticity to allow for flexion and stretching, while the conductive paste 300 keeps the electrical contact functional. The other end of the FPCB is connected to an external hardware via standard connectors or cables. The overall thickness of the interconnection system is thus only limited by the thickness of the soft substrate and encapsulation as the fingers lie inside the wells.

Although in this demonstration the soft device is manufactured using processes adapted from the semiconductor industry, this technique can be applied to any encapsulation material that can be machined with the required well/wall structures. The thicknesses of the substrate and encapsulation are in this example both equal to 200 μm. The pad-to-pad pitch can be in the order of a few hundred microns, for example 500 μm, and is limited by the resolution of the patterning/machining processes. The length of the channel (the well 202) is about 1 mm. The total thickness of the FPCB may be of about 100 μm depending on the manufacturing process. The thickness of the metal thin film is 23 nm. This technique is independent of the metallisation materials, provided that a compatible conductive paste is available.

This connection structure can be scaled up in number of channels as well as scaled down in size, as this only depends on the resolution of the metal trace (by lithography) and substrate patterning (laser machining). Additionally, the flexible printed circuit board can either be extended as a narrow strip used as a flat ribbon cable carrying all the channels, or terminated with small wires that can be soldered and bundled together using conventional techniques (as the limitations posed by soft materials do not apply). Furthermore, small active electronic chips can be integrated using standard electronic packaging techniques near the finger, so as to provide the device with enhanced functionality.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

1. An electrical interconnection system, comprising:
an interconnection board including an intrinsically non-elastic substrate, said intrinsically non-elastic substrate having a first face and an opposed second face, and at least one conductive track on or within at least a portion of said substrate;
a stretchable interconnect including an intrinsically elastic substrate, said intrinsically elastic substrate including at least one well or groove having at least one conductive element therein, said at least one well or groove being configured to accommodate said at least one conductive track of said interconnection board; and
at least one bolus of an electrically conductive paste located within said at least one well or groove, configured to electrically connect said at least one conductive element with said at least one conductive track.

2. The electrical interconnection system according to claim 1, wherein said at least one bolus of an electrically conductive paste is substantially composed of an adhesive elastic polymer configured to mechanically connect said at least one conductive element with said at least one conductive track.

3. The electrical interconnection system according to claim 1, wherein the intrinsically non-elastic substrate of the interconnection board is substantially composed of a flexible material.

4. The electrical interconnection system according to claim 1, wherein said intrinsically non-elastic substrate is planar at the interconnection site.

5. The electrical interconnection system according to claim 1, wherein the at least one conductive track of the interconnection board is located on an elongated member of the intrinsically non-elastic substrate.

6. The electrical interconnection system according to claim 5, wherein said elongated member of the intrinsically non-elastic substrate is planar.

7. The electrical interconnection system according to claim 5, wherein the interconnection board includes an array of elongated members, each including at least one conductive track.

8. The electrical interconnection system according to claim 7, wherein said stretchable interconnect includes an array of wells or grooves, each well or groove including one of said at least one conductive element therein.

9. The electrical interconnection system according to claim 1, wherein said at least one well or groove is configured to entirely accommodate said at least one conductive track of said interconnection board so that said at least one conductive track is completely embedded within said at least one bolus of electrically conductive paste.

10. The electrical interconnection system according to claim 1, wherein the at least one conductive element of the stretchable interconnect includes a stretchable metallic thin film.

11. The electrical interconnection system according to claim 1, wherein said at least one conductive element of the stretchable interconnect is embedded within said intrinsically elastic substrate.

12. The electrical interconnection system according to claim 1, wherein said bolus of electrically conductive paste includes a blend of a soft polymeric material and a plurality of conductive micro- or nano-particles, tubes wires or sheets.

13. The electrical interconnection system according to claim 1, further comprising an encapsulation layer of an adhesive and electrically insulating material encapsulating said opposite second face of the interconnection board and at least a portion of said stretchable interconnect including at least a portion of said at least one well or groove.

14. The electrical interconnection system according to claim 13, wherein said encapsulation layer is substantially composed of an intrinsically elastic material.

15. The electrical interconnection system according to claim 1, wherein said at least one conductive track or said at least one conductive element includes one end configured to be electrically connectable to an external device.

16. An article of manufacture comprising the electrical interconnection system according to claim 1.

17. The article of manufacture according to claim 16, wherein it the article of manufacture is a biomedical device configured to be temporarily or permanently implanted into a subject's body.
